(19) United States
(12) Patent Application Publication
(10) Pub. No.: US 2018/0273997 A1
(43) Pub. Date: Sep. 27, 2018

(54) CELLULOLYTIC ENZYME COMPOSITION

(71) Applicant: DESIGNER ENERGY, Rehovot (IL)

(72) Inventors: Alon KARPOL, Tel-Mond (IL); Ely MORAG, Rehovot (IL); Tal BARAK, Gedera (IL)

(73) Assignee: DESIGNER ENERGY, Rehovot (IL)

(21) Appl. No.: 15/757,094

(22) PCT Filed: Sep. 1, 2016
(86) PCT No.: PCT/IB2016/055246

§ 371 Date: Mar. 2, 2018

Related U.S. Application Data
(60) Provisional application No. 62/214,428, filed on Sep. 4, 2015.

(51) Int. Cl. C12P 19/14; C12N 1/20; C12N 9/42

(52) U.S. Cl. CPC C12P 19/14; C12P 2203/00; C12N 9/2445; C12N 1/20

(57) ABSTRACT

Compositions comprising an inactive cellulosome-producing microorganism and detectable extra-cellular osidase are provided. Compositions comprising an inactive Caldicellulosiruptor bescii and detectable extra-cellular beta-glucosidase or extra-cellular beta-xylosidase are also provided.

### CROSS-REFERENCE TO RELATED APPLICATION

The present application claims the benefit under 35 U.S.C. § 119(e) of U.S. Provisional Application No. 62/214,428 filed Sep. 4, 2015, the disclosure of which is expressly incorporated by reference herein in its entirety.

### FIELD OF THE INVENTION

The present invention relates to enzyme preparations and methods for use in the degradation of cellulosic biomass, as well as processes for producing such enzyme preparations.

### BACKGROUND

Plant cell wall components, primarily cellulose and hemicellulose, offer an excellent source of carbon and energy. Producing ethanol from plant materials, for example, requires three consecutive steps: physiochemical pretreatments, hydrolysis of cellulose and hemicelluloses into soluble sugars, and fermentation into ethanol. The most challenging, time consuming and costly process is the hydrolysis step, and much effort has been given to reduce the cost of the treatment and make it efficient and cost effective. Lignocellulose, a matrix of cellulose, lignin and optionally hemicellulose, is also difficult to hydrolyze due to the high association of the cellulose with hemicellulose and surrounding lignin, which results in a highly ordered, tightly packed, crystalline structure.

Only a few microorganisms have acquired the capacity to degrade these structures into soluble sugars. For example, the thermophilic anaerobic bacterium Clostridium thermocellum exhibits one of the most highly efficient degradation systems termed “cellulosome”. The cellulosome is a multienzyme system thought to allow synergistic enzyme activity in close proximity to the bacterial cell. Cellulosomes are protuberances produced on the cell wall of cellulolytic bacteria when growing on cellulosic materials. These protuberances are stable enzyme complexes that are firmly bound to the bacterial cell wall but flexible enough to also bind tightly to microcrystalline cellulose. The cellulosome is comprised of a diversity of hydrolytic activities. Among them are endoglucanases that catalyze the hydrolysis of internal bonds inside the cellulose chain and randomly produce new chain ends; and exoglucanases that cleave the cellulose chain at the exposed ends to produce mainly cellobiose. Other enzymes include xylanases, carbohydrate esterases, pectin lyases and others.

Cellulosomal protein complexes are routinely used in two main ways. The first is as part of an enzyme-microbe assembly of live and growing bacteria (see, for example, Lu et al., 2006, Enzyme-microbe synergy during cellulose hydrolysis by Clostridium thermocellum. Proc Natl Acad Sci USA 103:16165-9). In this method the bacteria are typically grown at the optimal conditions for their growth on lignocellulosic material, and the soluble sugars produced form the cell-associated and detached cellulases are used for yeast fermentation or are directly converted into ethanol by the bacteria. The second method involves extraction and isolation of sheared cellulosomal complexes in the bacterial medium during stationary phase of growth. The isolated cellulosomal complexes are then used for the degradation of cellulosic material. Both these methods have several limitations, for example, in the amount of biomass loading, bacteria:biomass ratios and high sensitivity to inhibitors.

Most of the research on cellulosomes was done on cell-free cellulosomal components. Very little is known about the potential activity of cell-bound cellulosomes produced with natural substrates, as the work that has been published so far has been limited.

For example, Zhang et al disclose use of cells growing on Cellobiose or Avicel (Zhang et al., 2003. “Quantification of cell and cellulase mass concentrations during anaerobic cellulose fermentation: development of an enzyme-linked immunosorbent assay-based method with application to Clostridium thermocellum batch cultures.” Anal Chem 75:219-27).

WO 2014/108900 and U.S. Provisional Application No. 61/750,827, which are incorporated by reference herein in their entireties, describe use of unprocessed cell pellets of a microorganism. However, there still remains a need for improved degradation of biomass, especially recalcitrant cellulosic biomass.

### DETAILED DESCRIPTION OF THE INVENTION

The particulars shown herein are by way of example and for purposes of illustrative discussion of the various embodiments of the present invention only and are presented in the cause of providing what is believed to be the most useful and readily understood description of the principles and conceptual aspects of the invention. In this regard, no attempt is made to show details of the invention in more detail than is necessary for a fundamental understanding of the invention, the description making apparent to those skilled in the art how the several forms of the invention may be embodied in practice.

The present invention will now be described by reference to more detailed embodiments. This invention may, however, be embodied in different forms and should not be construed as limited to the embodiments set forth herein. Rather, these embodiments are provided so that this disclosure will be thorough and complete, and will fully convey the scope of the invention to those skilled in the art.

Unless otherwise defined, all technical and scientific terms used herein have the same meaning as commonly understood by one of ordinary skill in the art to which this invention belongs. The terminology used in the description of the invention herein is for describing particular embodiments only and is not intended to be limiting of the invention. As used in the description of the invention and the appended claims, the singular forms “a,” “an,” and “the” are intended to include the plural forms as well, unless the context clearly indicates otherwise.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should be construed in light of the number of significant digits and ordinary rounding approaches.

As used herein, the term “about”, when referring to a measurable value such as an amount, a temporal duration, and the like, is meant to encompass variations of +/−10%, more preferably +/−5%, even more preferably, +/−1%, and still more preferably +/−0.1% from the specified value, as such variations are appropriate to perform the disclosed methods.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements. Every numerical range given throughout this specification will include every narrower numerical range that falls within such broader numerical range, as if such narrower numerical ranges were all expressly written herein.

Additional advantages of the invention will be set forth in part in the description which follows, and in part will be obvious from the description, or may be learned by practice of the invention. It is to be understood that both the foregoing general description and the following detailed description are exemplary and explanatory only and are not restrictive of the invention, as claimed.

### I. Microorganism Growth

Microorganisms

As used herein, the term “cellulosome-producing microorganisms” refers to microorganisms (including bacteria and fungi) that produce cellulosome complexes. A cellulosome is a multi-enzyme complex of cellulases, hemicellulases and other carbohydrate-active enzymes. Its basic structure typically contains a non-catalytic subunit called scaffoldin that binds the insoluble cellulosic substrate via a cellulose-specific carbohydrate-binding module (CBM). The scaffoldin subunit typically contains a set of subunit-binding modules, termed cohesins, that mediate specific incorporation and organization of the various enzymatic subunits into the complex through a complementary binding module, termed dockerin that is present in each enzymatic subunit. The assembly of the enzymes into the complex ensures their collective targeting to a specific region of the substrate thereby facilitating stronger synergism among the catalytic components. The cellulosomes are typically bound to the cell surface of the microorganism but may also be secreted to the surrounding environment. For example, some cellulosome-producing bacteria release their cell-bound cellulosomes during the stationary growth phase. In addition to cellulosome complexes, a cellulosome-producing microorganism may also produce free, non-cellulosomal enzymes that degrade cellulosic substances.

The cellulosome-producing microorganisms utilized herein may include aerobic and anaerobic, thermophilic and mesophilic microorganisms. Each possibility represents a separate embodiment of the invention.

In some embodiments, the cellulosome-producing microorganism is a bacterium.

Examples of suitable bacteria include, but are not limited to, Clostridium thermocellum (anaerobic, thermophilic), Clostridium clariflavum (anaerobic, moderate thermophilic), Clostridium cellulolyticum (anaerobic, mesophilic), Clostridium cellulovorans (anaerobic, mesophilic), Clostridium josui (anaerobic, moderate thermophilic), Acetivibrio cellulolyticus (anaerobic, mesophilic), Bacteroides cellulosolvens (anaerobic, mesophilic) and a Ruminococcus species (anaerobic, mesophilic). Each possibility represents a separate embodiment of the invention.

In some embodiments, the cellulosome-producing microorganism is a fungus.

Examples of suitable fungi include, but are not limited to, a Neocallimastix species (anaerobic, mesophilic), a Piromyces species (anaerobic, mesophilic) and a Orpinomyces species (anaerobic, mesophilic). Each possibility represents a separate embodiment of the invention.

The term “non-cellulosome-producing microorganism” may refer to an anaerobic, thermophilic bacterium that secretes cellulases and hemicellulases in free form or that produces exposed extracellular cellulases and hemicellulases at the cell surface. Caldicellulosiruptor bescii is a cellulose-degrading microorganism that flourishes in very high temperatures—up to 80° C. This organism does not produce cellulosomes to hydrolyze cellulose, but rather secretes its soluble cellulases into the medium. The secreted cellulases produced by C. bescii are characterized by highly modulated proteins—with many different modules along the polypeptide chain.

During growth on crystalline cellulose, the thermophilic bacterium C. bescii secretes several cellulose-degrading enzymes. Among these enzymes is CelA (also known as CbCel9A/Cel48A), which is reported as the most highly secreted cellulolytic enzyme by this bacterium. CelA is a large multi-modular polypeptide, comprising an N-terminal catalytic glycoside hydrolase family 9 (GH9) module and a C-terminal GH48 catalytic module that are separated by a family 3c carbohydrate-binding module (CBM3c) and two identical CBM3bs.

Biomass

According to the principles of the present invention, the cellulosome-producing microorganisms are grown in the presence of a cellulosic material. As used herein, the terms “cellulosic materials” and “cellulosic biomass” are used interchangeably and refer to materials that contain cellulose, in particular materials derived from plant sources that contain cellulose. Cellulose is a linear polysaccharide polymer composed of 13-1,4 linked D-glucose molecules. It is the major component of plant cell walls.

In some embodiments, the cellulosic material is purified cellulose. In some embodiments, the cellulosic material is microcrystalline cellulose. As used herein, “microcrystalline cellulose” refers to purified, partially depolymerized cellulose prepared by treating alpha cellulose. Examples of microcrystalline cellulose include microcrystalline cellulose sold under the trade name Avicel®.

In other embodiments, the cellulosic material is a complex cellulosic material which comprises cellulose and additionally one or more complex polysaccharides or other polymers, typically polysaccharides and polymers of plant cell walls, such as hemicellulose and lignin.

As used herein, the term “hemicellulose” has its art-known meaning and refers to a group of branched polysaccharide hetero- or homo-polymers composed of a variety of sugar monomers. Hemicellulose includes, for example, xylan, glucuronoxylan, arabinoxylan, glucomannan, and xyloglucan, in complex branched structures with a spectrum of substituents.

As used herein, the term “lignin” has its art-known meaning and refers to a polymeric material, mainly composed of linked phenolic monomeric compounds, such as p-coumaryl alcohol, coniferyl alcohol, and sinapyl alcohol, which forms the basis of structural rigidity in plants and is frequently referred to as the woody portion of plants. Lignin is also considered to be the non-carbohydrate portion of the cell wall of plants.

In some embodiments, the cellulosic material comprises cellulose, hemicellulose, and lignin. A cellulosic material containing cellulose, hemicellulose and lignin may be referred to as a “ligno-cellulosic material”. In some embodiments, the cellulosic material is ligno-cellulosic.

The cellulosic material may include natural plant biomass and also paper waste and the like. Examples of suitable cellulosic materials include, but are not limited to, wheat straw, switchgrass, corn cob, corn stover, sorghum straw, cotton straw, bagasse, energy cane, hard wood paper, soft wood paper, and combinations thereof. Each possibility represents a separate embodiment of the invention.

Additional examples include plant biomass of a Populous species, a Salix species, an Acacia species, a Tamarix species, Arundo donax, Miscanthus giganteus and combinations thereof. Each possibility represents a separate embodiment of the invention. The plant biomass may be derived from stems, leaves, hulls and husks.

Pre-Treatment

In some embodiments, the cellulosic material is pre-treated prior to its utilization for the microorganisms growth, to increase its susceptibility to hydrolysis. Pre-treatment may include chemical and physical pre-treatments or combinations thereof, and can be performed by methods known in the art. Exemplary pre-treatment procedures are provided in the Examples section below.

Physical pre-treatment techniques include, for example, various types of milling/comminution (reduction of particle size), irradiation, steaming/steam explosion, and hydrothermolysis.

Chemical pre-treatment techniques include, for example, acid, dilute acid, base, organic solvent, lime, ammonia, sulfur dioxide, carbon dioxide, pH-controlled hydrothermolysis, wet oxidation, and solvent treatment.

Acid pre-treatments are typically performed using sulfuric acid. Other acids may be used, such as hydrochloric acid, phosphoric acid, nitric acid or any mixture thereof. In general, the acid is usually mixed or contacted with the cellulosic material and the mixture is held at a temperature in the range of about 25-180° C. for a period ranging from about 1-60 minutes.

Alkali pre-treatments may be performed using sodium hydroxide, ammonia, calcium hydroxide and potassium hydroxide. In general, the base is usually mixed or contacted with the cellulosic material and the mixture is held at a temperature in the range of about 0-130° C. for a period ranging from about 5-300 minutes.

A particular example for a chemical pretreatment includes mercerization. An exemplary procedure is provided below.

Composition of the Growth Medium

The above described cellulosic materials are added to the growth medium of the microorganisms. The culture medium is typically an aqueous medium. In some embodiments, the medium contains about 0.1-25% cellulosic material or any amount therebetween, for example about 0.5-15% or any amount therebetween, about 0.5-5% or any amount therebetween. Each possibility represents a separate embodiment of the invention. As defined herein, the concentration of the cellulosic material in the medium is a ratio by weight of dry cellulosic material to a culture medium volume.

In some embodiments, the culture medium further comprises one or more complex plant polysaccharides, such as pectin. Suitable amounts of pectin are in the range of about 0.5-10 g/l.

The culture medium typically further comprises nitrogen and phosphate sources, and may also include appropriate salts, minerals, metals and other nutrients as known to a person of skill in the art.

Nitrogen sources may include, for example, ammonia, such as ammonium sulfate, and peptides. A nutrient source containing protein hydrolysate and amino acids such as yeast extract and peptone may also be used.

Examples of minerals include minerals and mineral salts that can supply nutrients comprising P, K, Mg, S, Ca, Fe, Zn, Mn, and Cu.

Further, for suppressing pH decrease due to the proliferation, a buffering agent may be added.

In the case of anaerobic fermentation, a gas phase of the culture medium may be substituted by carbon dioxide gas or nitrogen gas.

The above media components may be added prior to, simultaneously with or shortly after inoculation of the culture with the microorganism.

Culture Conditions

Culture conditions may be set up according to optimum temperature and optimum pH of the particular microorganism used. In addition, culture is performed under aerobic or anaerobic conditions, according to the nature of the microorganism used. The optimal pH and temperature for each microorganism, as well as information regarding the microorganism being aerobic or anaerobic, can be readily found in the scientific literature, for example, publications and information available from the DSMZ (Deutsche Sammlung von Mikroorganismen and Zellkulturen) GmbH, Braunschweig Germany.

The culturing process according to the present invention is typically a batch process. In a batch process, all the necessary materials, with the exception of oxygen for aerobic processes, are placed in a reactor at the start of the operation and the fermentation is allowed to proceed until completion, at which point the product is harvested. The microorganisms can be cultured in conventional fermentation bioreactors. A starter culture can be grown prior to culturing in bioreactors, as known in the art. The obtained started culture is than inoculated to a bioreactor.

The microorganisms are typically grown until late exponential phase or stationery phase, for example early stationery phase (e.g. about 12-24 hr), or late stationery phase (e.g., about 24 hours after the stationery phase begins).

In order to identify the late exponential phase or early stationary phase, several methods can be applied, such as CO₂ production, cell density, pH change and more. For example, in regard to CelZyme production the use of a base (alkaline chemical) consumption may be used. In certain embodiments, methods for detecting late exponential phase or stationary phase, such as OD measurement may not be applicable due to high solid load or the inability to perform such a measurement in real time or on-line). Once the base consumption is leveled (reaches a plateau) the fermentor may be shut down.

In some embodiments, the microorganism are cultured for a period of time ranging from about 12-100 hours or any amount therebetween, for example from about 24-72 hours, from about 24-48 hours, or any amount therebetween.

The exact duration of incubation can be determined by one or more of the following:
NaOH consumption—NaOH is added to the culture medium in order to neutralize an acid formed during growth of the microorganisms. Fermentation is stopped and the pellet is collected when NaOH is no longer consumed, or about 24 hours after NaOH is no longer consumed.
Preliminary assay—a preliminary assay can be performed in order to determine the incubation duration in which the highest level of cellulolytic activity is obtained from the collected pellet. The preliminary assay includes the following steps: (i) culturing a cellulosome-producing bacteria of choice in a medium containing a cellulosic substrate of choice; (ii) sampling the culture at various time points: for example, samples may be taken out every 6-10 hours, or at particular time points, such as 12 hours, 18 hours, 24 hours, 36 hours, 60 hours and 72 hours after the beginning of incubation; (iii) testing each sample for its cellulolytic activity (for example, on microcrystalline cellulose); (iv) identifying the sample with the maximum cellulolytic activity, and accordingly the time point in which maximum activity is obtained may be selected. This time point would be the optimal incubation duration for the particular microorganism in the particular medium. This incubation duration should be used for preparing the enzymatic compositions of the present invention.

Continuous Vs. Batch Fermentation

In batch cultivation, the bacteria are inoculated into the bioreactor (always stirred tank bioreactor). Then, under certain conditions (temperature, pH, aeration, etc.) the bacteria go through all the growth phases (lag, exponential, stationary). At last, the fermentation is stopped and the product is collected. Then, after cleaning and sterilization of the fermenter, the fermenter is ready for another batch.

In continuous cultivation, the fresh medium flows into the fermentor continuously, and part of the medium in the reactor is withdrawn from the fermenter at the same flow rate of the inlet flow. The continuous fermentation for CelZYme production should start with regular inoculation of the fermentor with a starter, and then culture is left to grow until the desired point (late exponential or early logarithmic). Once the culture reaches the specific point, continuous mode is applied. In the case of CelZyme production, in-goes the fresh medium with nutrients and a new carbon and energy source, and out-comes the exhausted medium with the newly produced CelZyme, which will be collected to downstream process if necessary, or immediately to packaging and storage.

### II. Cellulolytic Enzyme Preparation

Pellet Composition and Characterization

The collected pellet fraction comprises insoluble components that were present in the culture broth. These components include cells and/or cell debris, cell-bound cellulosomes, residual un-hydrolyzed or partially hydrolyzed cellulosic biomass, and biomass-bound cellulolytic enzymes.

The cells present in the pellet are inactivated according to the principles of the present invention. In some embodiments, when anaerobic microorganisms are used, they are inactivated upon exposure to oxygen. Thus, inactivating anaerobic microorganisms typically occurs during the collection and processing of the culture broth to obtain a pellet. For aerobic microorganisms, inactivation may be performed, for example, by exposing the cells to an azide, such as sodium azide or by sonication. In some embodiments, inactivation of aerobic microorganisms is performed by a method other than sonication. In some embodiments, inactivation of the microorganisms is performed without affecting cell integrity. Typically, at least 80% of the cells are inactivated, for example at least 85%, at least 90%, at least 95%, or 100% of the cells are inactivated.

According to the principles of the present invention, the compositions may comprise an unprocessed pellet that has not been subjected to purification processes to isolate or remove particular components from the pellet, including for example various chromatography procedures to isolate enzymatic components, or sonication and centrifugation to separate and remove cells from the composition.

In some embodiments, the pellet is subjected to processing other than purification of enzymes or other components of the pellet.

The pellet may be used as a wet preparation, or can be dried by methods known in the art such as spray-drying or lyophilization.

Thus, in some embodiments, the compositions of the present invention comprise a wet pellet of a cellulosome-producing microorganism. In some embodiments, the wet pellet is characterized by water content in the range of 60-80% or any amount therebetween, for example about 65-75% water or any amount therebetween. Each possibility represents a separate embodiment of the invention. Determination of the water content may be performed by any method known to a person of skill in the art, such as determining mass loss following heating or oven drying.

In other embodiments, the compositions of the present invention comprise a dried pellet of a cellulosome-producing microorganism. In some embodiments, the dried pellet comprises about 3-20% residual water, for example about 3-10%, or about 3-5% residual water.

The pellet (wet or dry) may be characterized by determining one or more of the following:
Protein content: may be determined, for example, using colorimetric methods, such as the bicinchoninic acid (BCA) protein assay. The protein content is calculated using a calibration curve of a known protein (e.g. bovine serum albumin).
Biomass content: the biomass to be calculated may include cellulose, hemicelluloses and lignin. Exemplary methods for determining ligno-cellulosic content, total carbohydrate content and/or cellulose and hemicellulose content are provided in the Examples section herein below.
DNA content: may be determined, for example, using spectrophotometry analysis at 260 nm.

In some embodiments, the pellet is characterized by a protein:biomass ratio in the range of about 3:1-1:5 (w/w).

In some embodiments, the pellet is characterized by a DNA:biomass ratio in the range of about 1:400-1:25 (w/w), for example about 1:300-1:100.

The pellet preparation of the present invention has a cellulolytic activity, i.e., it is capable of hydrolyzing cellulosic substrates. An exemplary assay for determining the ability of the preparation to degrade cellulosic substrates is provided in the Examples section hereinbelow.

The enzyme content of the pellet preparation includes a variety of enzymatic activities. In particular, the preparation comprises a variety of carbohydrate active enzymes. As used herein, the term “carbohydrate active enzyme” refers to an enzyme that catalyzes the breakdown or modification of carbohydrates and glycoconjugates. The broad group of carbohydrate active enzymes is divided into enzyme classes and further into enzyme families according to a standard classification system (Cantarel et al. 2009 Nucleic Acids Res 37:D233-238). According to this classification system, four enzyme classes are defined, namely glycoside hydrolases, glycosyl transferases, polysaccharide lyases and carbohydrate esterases. An informative and updated classification of carbohydrate active enzymes is available on the Carbohydrate-Active Enzymes (CAZy) server (www.cazy.org) and/or CAZypedia (www.cazypedia.org).

In some embodiments, the pellet preparation comprises at least one endocellulase and at least one exocellulase.

In some embodiments, the pellet preparation comprises at least one hemicellulase. In some embodiments, the hemicellulase is selected from the group consisting of a xylanase, and arabinofuranosidase, an acetyl xylan esterase, a glucuronidase, an endo-galactanase, a mannanase, an endo-arabinase, an exo-arabinase, an exo-galactanase, a ferulic acid esterase, a galactomannanase, a xylogluconase, and mixtures thereof.

Beta-glucosidase

In some embodiments, the compositions of the present invention comprise a beta-glucosidase. As used herein, the term “β-glucosidase” refers to an enzyme that hydrolyzes terminal, non-reducing β-D-glucose residues from cello-oligodextrins. In particular, this type of enzyme cleaves cellobiose to generate two molecules of glucose.

In some embodiments, the beta-glucosidase originates from the cellulosome-producing microorganism, meaning that it is endogenously produced by the microorganism. The beta-glucosidase, according to some embodiments, is a recombinant enzyme that is expressed and secreted by the microorganism, which has been genetically modified to produce it. In some embodiments, the beta-glucosidase is incorporated into the cellulosome of the microorganism. Incorporation of the enzyme into the cellulosome can be achieved, for example, via a dockerin present in the beta-glucosidase and a matching cohesin present in a scaffoldin subunit of the cellulosome, or vice versa, namely, via a cohesin present in the beta-glucosidase and a matching dockerin present in a scaffoldin subunit (type-II interaction). In additional embodiments, the beta-glucosidase comprises a cellulose-binding module.

The beta-glucosidase according to these embodiments is extra-cellular and detectable, meaning that it is secreted from the microorganism cells and it is possible to detect its activity after collection of the pellet. Detection of beta-glucosidase activity is typically performed using p-Nitrophenyl-beta-D-glucoside (pNPG) or cellobiose as a substrate by assays known to persons of skill in the art.

In other embodiments, the beta-glucosidase is exogenous, meaning that it is added to the composition exogenously rather than produced by the microorganism. The exogenously added beta-glucosidase may be a commercially available beta-glucosidase, available, for example, from Codexis Inc. Alternatively, it may be prepared by means of well-known recombinant methods, or by classical purification methods. Sources of beta-glucosidase suitable for use in accordance with the present invention include bacterial and fungal sources. In some embodiments, an exogenous beta-glucosidase is added to a composition which includes an unprocessed pellet of a microorganism that naturally produces a beta-glucosidase as a native enzyme. For example, it may be added to a composition containing an unprocessed pellet of a microorganism that produces an intracellular beta-glucosidase (which is undetectable in the pellet preparation).

In some embodiments, the exogenously added beta-glucosidase is a thermostable beta-glucosidase. In some embodiments, the beta-glucosidase is thermostable in temperatures above 50° C., above 60° C., for example above 70° C., in the range of 60-80° C. Each possibility represents a separate embodiment of the invention.

In some embodiments, the exogenously added beta-glucosidase is characterized by an optimum temperature for activity above 50° C., for example above 60° C., above 70° C., in the range of 60-80° C. Each possibility represents a separate embodiment of the invention.

In some embodiments, the beta-glucosidase originates from a thermophilic microorganism. In the case of a beta-glucosidase that is obtained from a thermophilic microorganism, the optimum temperature for the enzyme is usually the optimal growth temperature of the microorganism.

Examples of suitable sources of thermostable beta-glucosidases in the temperature optimum range of 60-80° C. include: Agrobacterium tumefaciens, Aspergillus aculeatus, Aspergillus foetidus, Aspergillus fumigatus, Aspergillus japonicas, Aspergillus niger, Aspergillus oryzae, Aspergillus pulverulentus, Aspergillus pulverulentus YM-80, Aspergillus tubingensis, Aspergillus wentii, Athelia rolfsii, Aureobasidium pullulans, Caldicellulosiruptor saccharolyticus, Cellvibrio gilvus Cellulomonas biazotea, Ceriporiopsis subvermispora, Ceriporiopsis subvermispora CS-1, Chaetomium thermophilum, Citrus sinensis, Clostridium stercorarium, Clostridium thermocellum, Evemia prunastri, Fomitopsis palustris, Fusarium oxysporum, Hemicarpenteles omatus, Hordeum vulgare, Humicola sp., Hypocrea jecorina, Lenzites trabea, Melanocarpus sp., Melanocarpus sp. MTCC 3922, Millerozyma farinosa, Myceliophthora heterothallica, Paecilomyces sp. J18, Penicillium aurantiogriseum, Penicillium brasilianum, Penicillium brasilianum IBT 20888, Penicillium decumbens, Penicillium purpurogenum, Penicillium purpurogenum KJS506, Penicillium verruculosum, Periconia sp., Phoma sp., Physarum polycephalum, Pyrococcus furiosus, Rhizomucor miehei, Sclerotinia sclerotiorum, Sulfolobus solfataricus, Talaromyces emersonii, Talaromyces emersonii CBS 814.70, Termitomyces clypeatus, Thermoascus aurantiacus, Thermobispora bispora, Thermomyces lanuginosus, Thermotoga maritime and Thermus thermophiles (found in BRENDA database, available at: www.brenda-enzymes.org).

In some embodiments, the beta-glucosidase is from a bacterial source. In other embodiments, the beta-glucosidase is from a fungal source.

Particular, non-limiting, examples include Clostridium thermocellum, e.g. BglA, and Thermoanaerobacter brockii, e.g., CglT.

In some embodiments, the amount of the exogenously added beta-glucosidase in the composition is in the range of about 0.001-10% (w/w) or any amount there between, for example about 0.01-5% (w/w) or any amount there between. Each possibility represents a separate embodiment of the invention.

In some embodiments, the β-glucosidase is classified in a glycoside hydrolase family selected from the group consisting of family 1, 3, 9, 30 and 116. Each possibility represents a separate embodiment of the invention.

Beta-Xylosidase

In some embodiments, the compositions of the present invention comprise a beta-xylosidase. As used herein, the term “β-xylosidase” or with system name 4-beta-D-xylan xylohydrolase refers to an enzyme that hydrolyzes the following chemical reaction Hydrolysis of (1→4)-beta-D-xylans, to remove successive D-xylose residues from the non-reducing termini. This enzyme also hydrolyses xylobiose.

Xylan 1,4-beta-D-xylosidase catalyzes hydrolysis of non-reducing end xylose residues from xylooligosaccharides. The enzyme is currently used in combination with beta-xylanases in several large-scale processes for improving hemicellulose hydrolysis.

These enzymes are exo-type glycosidases that remove xylose monomers from the non-reducing end of xylooligosaccharides and serve as one component of a multi-enzyme milieu that biodegrades hemicellulose. Thus far, β-xylosidase activity assays have relied mainly on the use of substrate analogs consisting of xylose O-linked to a chromophore or fluorophore, such as nitrophenyl or β-umbelliferyl.

The beta-xylosidase may be exogenous, meaning that it may be added to the composition exogenously rather than produced by the microorganism. The exogenously added beta-xylosidase may be a commercially available beta-xylosidase. Alternatively, it may be prepared by means of well-known recombinant methods, or by classical purification methods. Sources of beta-xylosidase suitable for use in accordance with the present invention include bacterial and fungal sources.

The β-xylosidase can be added as a free enzyme without any module or other domain attached to it. Or it can be manipulated so as to add, for example, a dockerin module to its C-terminus or N-terminus. The dockerin module will direct the β-xylosidase into the cellulosomal fraction of the CelZyme, thus targeting the β-xylosidase into close proximity of the hydrolysis of the hemicellulose.

The amount of the exogenously added β-xylosidase in the composition is in the range of about 0.001-10% (w/w) or any amount there between, for example about 0.01-5% (w/w) or any amount there between.

The β-xylosidase is classified in a glycoside hydrolase family selected from the group consisting of family 1, 3, 31, 39, 43, 51, 52, 116, 120.

### III. Cellulose Hydrolysis by the Preparation

The compositions of the present invention may be utilized for hydrolysis of cellulosic substrates, including ligno-cellulosic substrates.

By the term “cellulosic substrate”, it is meant any substrate that contains cellulose, in particular substrates derived from plant sources that contain cellulose, examples of which were provided hereinabove. The term “cellulosic substrate” further encompasses hemicellulose-containing substrates and ligno-cellulosic substrates.

The “cellulosic substrate” or cellulosic material for hydrolysis may be pre-treated or utilized in crude (untreated) form. Nonetheless the pre-treatment process for each cellulosic substrate may be different. In an embodiment the cellulosic substrate or material may originally be derived from the same biomass source or have the same origin, but may be treated later under varying conditions. For example, a first cellulosic substrate for CelZyme production may require less severe pre-treatment conditions (lower temperature, reduced acid or base concentration) compared to a second cellulosic substrate pre-treatment which will be used for the hydrolysis by the enzyme.

In some embodiments, the concentration of the cellulosic substrate in the reaction mixture is about 5% or more, for example about 10% or more, in the range of about 5-30% or any amount therebetween, for example about 10-20% or any amount therebetween. Each possibility represents a separate embodiment of the invention.

In some embodiments, the weight ratio between the cellulolytic composition of the present invention and the cellulosic substrate is in the range of about 2-20% or any ratio therebetween, for example about 5-15% or any ratio therebetween. Each possibility represents a separate embodiment of the invention.

The reaction mixture may be incubated for from about 4 hours to about 120 hours, or any amount therebetween, for example between about 20-100 hours or any amount therebetween, at a temperature from about 30° C. to about 80° C., preferably above 60° C., or any temperature therebetween. Each possibility represents a separate embodiment of the invention. The optimal pH and temperature depend on the microorganism used for the production of the enzyme preparation, as well as the type of beta-glucosidase or beta-xylosidase in the composition (if added).

By relieving end-product inhibition of endoxylanases and exo/endo-glucanases (such as xylobiose and cellobiose), it may be possible to further enhance the hydrolysis of the cellulosic material.

Following incubation, the reaction products can be used for further processing, for example as a substrate for producing ethanol, butanol, sugar alcohols, lactic acid, acetic acid, production of fuels, e.g., biofuels such as synthetic liquids or gases, such as syngas, or the end products may be concentrated and purified using standard methods as known in the art.

The degradation products typically comprise mono-, di- and oligosaccharides, including but not limited to glucose, xylose, cellobiose, xylobiose, ellotriose, cellotetraose, arabinose, and/or xylotriose.

### IV. Recycling the Enzymatic Complex

Recycling of the enzymatic preparation refers to the repeated use of the same enzymatic preparation for successive hydrolyses of cellulosic substrates. Recycling typically comprises contacting a first sample of a cellulosic substrate with the enzyme composition to obtain hydrolysis of the first sample of a cellulosic substrate into hydrolysis products; (ii) collecting the hydrolysis products; and (iii) contacting a second sample of a cellulosic substrate with the enzyme composition to obtain hydrolysis of the second sample of a cellulosic substrate into hydrolysis products.

The ability to recycle the enzymes for successive fermentations can substantially reduce process costs. Advantageously, the compositions of the present invention can be simply recycled by subjecting samples to brief centrifugation (or other means of separation such as filtration through a micro sieve membrane) and replacing the supernatant fluids with fresh cellulosic substrate.

### V. Examples

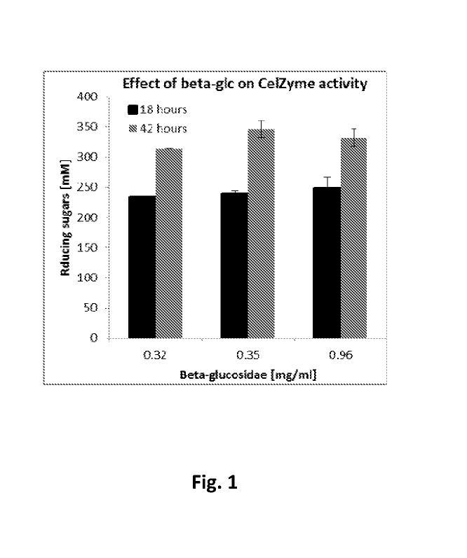

### Example/FIG. 1

CelZyme activity was tested for optimal condition by increasing the beta-glucosidase concentrations. As a source for beta-glucosidase, the CglT enzyme from thermoanaerobacter brockii, was used. Corn stover, pretreated with acid, was used as the model substrate at 15% biomass loading. CelZyme loading was 50% of the cellulose content of the feedstock and the CglT final concentration ranged between-0.32 mg/ml to 0.96 mg/ml. Hydrolysis temperature was set to 70 C, with the appropriate buffer. Samples were taken after 18 hours and 42 hours. Each data represent a triplicate.

As can be seen in FIG. 1, there is no major difference in reducing sugars concentration when using 0.35 mg/ml of CglT compare to 0.96 mg/ml. The amount of sugars in the hydrolysate was 350 mM which represent 64% hydrolysis yield.

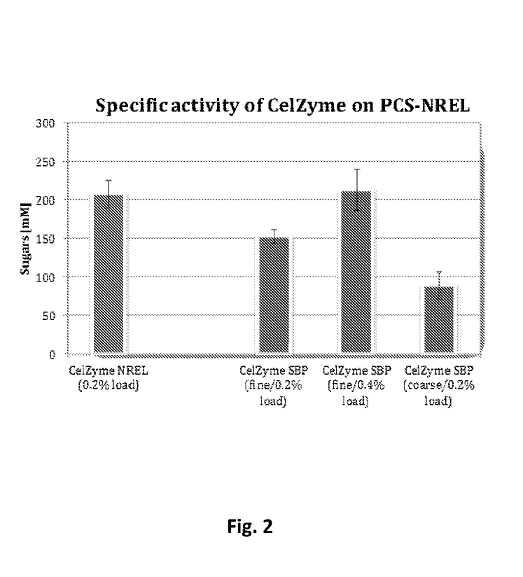

### Example/FIG. 2

In this experiment, we have tested the ability of the sugar beet pulp to support CelZyme production in small scale as a source for carbon and energy. The sugar beet pulp was dried and either milled and then sieved through 60 mesh (fine), or the remaining milled sugar beet pulp that was left on the sieve was also used (coarse).

The Beet pulp was used as a source for energy and carbon in the growth medium with loading 0.2% or 0.4%. The growth bottle were than inoculated with C. thermocellum and the bottles were incubated for 24 hours at 60 C. The resulting CelZyme was tested on Pretreated Corn stover (PCS-NREL) which is used a standard assay for CelZyme hydrolysis. The sugar concentration from the CelZyme^{SBP}/0.4% load/fine was similar to the sugar concentration released by the standard CelZyme^{NREL}˜210 mM. CelZyme produced from 0.2% load with fined milled or 0.2% with coarse mill, showed 25%-35% reduction in activity.

### CONCLUSIONS

SBP can support CelZyme^{SBP} production, and the resulting CelZyme^{SBP} is competent as the CelZyme^{NREL}.

1. A solid cellulolytic enzyme composition comprising an inactive Caldicellulosiruptor bescii and detectable extra-cellular beta-glucosidase.

2. The composition of claim 1, further comprising at least cellulose on a dry basis.

3. The composition of claim 2, wherein a protein:cellulose ratio of the solid cellulolytic enzyme composition is in the range of about 6:1-1:100 (w/w).

4. The composition of claim 2, wherein a DNA:cellulose ratio of the solid cellulolytic enzyme composition is in the range of 1:800-1:10 (w/w).

5. The composition of claim 1, further comprising at least 2% lignin on a dry basis.

6. The composition of claim 1, further comprising at least 1% hemicellulose on a dry basis.

7. The composition of claim 1, further comprising a cellulosome-producing microorganism.

8. The composition of claim 7, wherein the cellulosome-producing microorganism is an anaerobic, thermophilic bacterium selected from the group consisting of Clostridium thermocellum, Clostridium clariflavum, and Clostridium josui.

9. The composition of claim 7, wherein the cellulosome-producing microorganism is an anaerobic, mesophilic bacterium, selected from the group consisting of Clostridium cellulolyticum, Clostridium cellulovorans, Acetivibrio cellulolyticus, Bacteroides cellulosolvens and a Ruminococcus species.

10. The composition of claim 7, wherein the cellulosome-producing microorganism is an anaerobic, mesophilic fungus, selected from the group consisting of a Neocallimastix species, a Piromyces species and an Orpinomyces species.

11. A solid cellulolytic enzyme composition comprising at least one of an inactive cellulosome-producing microorganism and an inactive Caldicellulosiruptor bescii, and further comprising detectable extra-cellular beta-xylosidase.

12. The composition of claim 11, further comprising at least 2% cellulose on a dry basis.

13. The composition of claim 12, wherein a protein:cellulose ratio of the solid cellulolytic enzyme composition is in the range of about 6:1-1:100 (w/w).

14. The composition of claim 12, wherein a DNA:cellulose ratio of the solid cellulolytic enzyme composition is in the range of 1:800-1:10 (w/w).

15. (canceled)

16. The composition of claim 11, further comprising at least 1% hemicellulose and/or at least 2% lignin on a dry basis.

17. The composition of claim 11, wherein the cellulosome-producing microorganism is an anaerobic, thermophilic bacterium selected from the group consisting of Clostridium thermocellum, Clostridium clariflavum, and Clostridium josui.

18. The composition of claim 11, wherein the cellulosome-producing microorganism is an anaerobic, mesophilic bacterium, selected from the group consisting of Clostridium cellulolyticum, Clostridium Cellulovorans, Acetivibrio cellulolyticus, Bacteroides cellulosolvens and a Ruminococcus species.

19.-21. (canceled)

22. A process for hydrolyzing a cellulosic material comprising: (i) culturing at least one of a cellulosome-producing microorganism and a Caldicellulosiruptor bescii in a growth medium comprising a first cellulosic material; (ii) removing solids present in the growth medium to obtain a solid preparation comprising extracellular heta-xylosidase or C. bescii; (iii) forming an hydrolysis medium comprising said solid preparation and a second cellulosic material; and (iv) allowing said solid preparation to hydrolyze said second cellulosic material in said hydrolysis medium for at least 1 hour to form a water soluble hydrolysate.

23.-40. (canceled)

41. A process of producing an enzymatic composition comprising (i) pre-treating cellulosic material, (ii) culturing Caldicellulosiruptor bescii in a growth medium comprising the pre-treated cellulosic material, and (iii) separating solids present in the culture medium to form an enzymatic composition.

42. The process of claim 41, further comprising inactivating the C. bescii.

43.-44. (canceled)
